(19) United States
(12) Patent Application Publication
(10) Pub. No.: US 2009/0162294 A1
(43) Pub. Date: Jun. 25, 2009

(54) DISPOSABLE INHALER

(76) Inventor: Johannes Werner, Halle (DE)

Correspondence Address: DAVID A. GUERRA;INTERNATIONAL PATENT GROUP, LLC, 2025 17TH AVENUE N.W., CALGARY, AB, T2M 0S7, CA

(21) Appl. No.: 11/719,875

(22) PCT Filed: Nov. 9, 2005
(86) PCT No.: PCT/DE2005/002004

§ 371 Date: May 22, 2007

(30) Foreign Application Priority Data
Nov. 22, 2004 (DE) 10 2004 056 309.8

(51) Int. Cl. A61K 9/72; A61P 43/00

(52) U.S. Cl. 424/40

(57) ABSTRACT

The inventive inhaler functions in a similar way to a cigarette but without the formation of carcinogenic substances and carbon monoxide, as instead of tobacco or other substances containing carbon, metallic components of a combustion body (3) are burnt. The air thus heated is used to evaporate the stimulant or active ingredient. The combustion metals are the physiological metals magnesium and iron or alloys of said metals comprising the elements sodium, potassium, calcium, zinc and phosphorus. The inventive disposable inhaler is used for the application of stimulants (nicotine) or medicinal active ingredients.

The invention relates to a disposable inhaler for inhaling active substances, wherein the active substances are incorporated in an active substance carrier, from which they are releasable by heat action.

Inhalation concerns the absorption of active substances into the human body through the lung. This type of active substance absorption serves for medicinal purposes on the one hand and, on the other hand, for the application of stimulants, and in this context particularly to provide the enjoyment of tobacco smoke. The method of enjoying tobacco smoke has remained unchanged for a long time. Thus, tobacco is slowly combusted in a pipe, a cigar or a cigarette. During such combustion the nicotine—the stimulant—contained in the tobacco is vaporized. In addition, there are formed during the combustion olfactors and flavoring substances as well as combustion residues which jointly with the stimulant, nicotine, are inhaled as tobacco smoke.

This manner of stimulant usage is harmful to the human body, since it is supplied by way of the lung not only with the stimulant nicotine and possibly olfactors and flavoring substances, but also harmful combustion residues. In this context the nicotine is one of the least harmful components of tobacco smoke, even though this active substance causes contraction, in particular of peripheral blood vessels and may thereby result in reduced blood supply and accordingly of oxygen supply to parts of the body. This disadvantage of the stimulant nicotine is reversible, however, because it is cancelled entirely once the absorbed nicotine has been eliminated from the body. Moreover, where the consumer enjoys a condition of general good health and if consumption is moderate, no damage is suffered.

Much more serious are the carcinogenic combustion residues of various substance classes, such as e.g. polycyclic aromatics, nitrosamines, aldehydes, aromatic amines and traces of carcinogenic metals (see also Table 1) which become absorbed during the inhaling of tobacco smoke. These components of tobacco smoke give rise to the problematic damaging effects, i.e. carcinoma of the lung and the respiratory system. In addition, the carbon monoxide formed during the combustion of tobacco must be mentioned as an important toxic agent. This toxic carbon monoxide causes a reduction of the fitness of the smoker by blocking the oxygen transport in blood. Damage to defectively blood supplied tissue regions due to oxygen starvation cannot be excluded in case of heavy consumption. An additional risk of damage to health results from the covering material of a cigarette. Here a cover of paper is used, the combustion of which results in additional carbon monoxide and carcinogenic substances.

Besides the inhalation of stimulants a medicinal variant of inhalation must also be considered, the application of medicinally active substances by way of the lung.

For such purposes, an inhalable vapor or aerosol must be produced for which, for example, an electrically operated nebulizer can be used. Such instruments are of complicated design and suitable only for the application of water soluble substances. They must be supplied with electrical energy. This by itself dictates a stationary use of the apparatus, e.g. in a hospital or at home. As a mobile variant of the application of water soluble substances a spray device is known. For medicinal substances which are not water soluble, but which can be vaporized by heating, an electrically heated vaporizer is known. Such instruments are only suitable for stationary use, since electrical current must be supplied for the vaporization. The “smoke-free” cigarette having the trade name “NicStic” may be considered a mobile variant of an electrically operated vaporizer, which was published under publication no. DE 10321379 A. In this case, the air is heated in an electrical device of cigarette size in order to vaporize an active substance (nicotine), for which purpose it is necessary however to carry along an energy storage device in the form of an accumulator.

From here the invention proceeds, which has made it as its object to avoid the aforesaid drawbacks and to provide a disposable inhaler which is capable of transporting the active substances, by way of inhalation into the lung, in spite of the combustion process, essentially without harmful combustion residues.

According to the invention, it is proposed in this context that the heating of the drawn in air proceeds by the combustion of an air-pervious combustion body which, as combustible components, contains metallic ingredients. In the subsidiary claims which follow advantageous embodiments of the disposable inhaler according to the invention are presented.

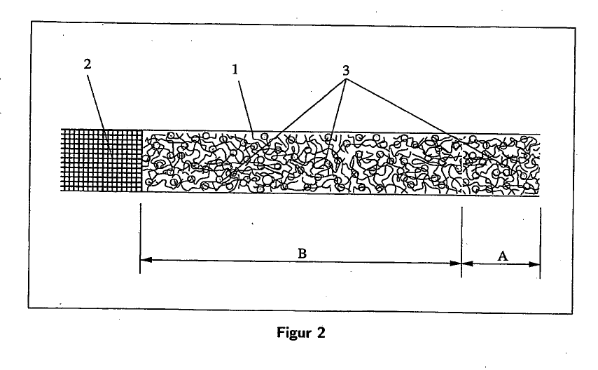

The heating of the air by combustion of metals provides the advantage that no carbonaceous substrates of natural or artificial origin are combusted. The combustion of metals can neither give rise to carbon monoxide nor to carcinogenic organic substances. Instead, the combustion product of metals or metal alloys takes the form of solid metal oxides in the form of ash. A minor portion thereof is entrained as fine dust by the air flow and reaches the respiratory organs in unfiltered form. Part of this metal oxide dust becomes bound by the mucus of the mucous membranes and is thus eliminated, the remainder being resorbed in the lung. Because the amount absorbed is very small even in the event of heavy consumption (the concentration of the dust in the inhaled air is below permissible maximum atmospheric concentration levels) no damage is suffered by the lung or other organs. A precondition for this is that the oxides are those of “physiological” metals. These metals or their compounds are naturally present in the body, have no toxic or carcinogenic effects and can be eliminated again by the body. In order to reduce even further the exposure of the metabolism due to inhaled metal oxide dusts, a filter is additionally used in certain working examples (see further below). Such filter may be an ordinary cigarette filter. Since with a disposable inhaler according to the invention the combustion residues are of a special kind, it is here also possible to employ special filters designed for such residues. In the case of a disposable inhaler having a cover of magnesium foil, and a combustion body composed of a magnesium wool-sand mixture (FIG. 2), the filter might be composed of sepiolith, a magnesium silicate. In Table 1 substances are listed which are formed when combusting a cigarette made of tobacco. These are contrasted against the substances which are formed when combusting a disposable inhaler according to the invention.

The disposable inhaler according to the invention is designed similar to a cigarette. In principle, it is composed of an air-pervious combustion body in the shape of a small rod. This, apart from the end faces, is enveloped by a cover or provided with a coating. The combustion body is composed of magnesium, pyrophorous iron, the alloys mentioned further below or contains these metals as combustible components. In accordance with one example, the combustion body acts simultaneously as the active substance carrier, in an alternative embodiment active substance carrier and combustion body are present as separate entities. Optionally, the combustion body may have added thereto, besides the combustion metal, additive components serving as combustion regulators, e.g. magnesium oxide or ceramic particles.

The active substances which are incorporated or applied either in or on the combustion body or which are present in separate active substance carriers, are evaporated by the air heated by the combustion of metal and passed on to the lung. The cover enclosing the active substance carrier may include a filter for the retention of metal oxide dust, arising during the combustion process. In the application as a source of stimulant the structure according to the invention contains as active substance pure nicotine and, optionally, a scent which enhances the odor and/or the flavor, but which is harmless for the smoker. For the use as a medicinal disposable inhaler the active substance carrier contains a medicinally active substance, which—like nicotine—evaporates during smoking and is inhaled into the lung in vapor form.

It was found to be advantageous for the invention that the combustion body is formed of metal foil, metal wire, metal wool or an inorganic, air-pervious structure, containing combustible metallic substances. As metals for the structure of the combustion body primarily the “physiological” metals magnesium and iron are to be considered. The metals sodium, potassium and calcium which likewise occur in the body react in their pure state rapidly with water with the formation of hydrogen. That also applies to alloys of other metals with a high content of sodium, potassium and/or calcium. Since during their combustion moreover oxides are formed which with water form strongly basic, caustic hydroxides, they can be used as components of alloys only in low proportions, e.g. below two percent. A further “physiological” metal which can be used as an alloying component is zinc. It suffers from the disadvantage that even at temperatures below 600 degrees Celsius a substantial part enters into the gaseous state and after reaction with air oxygen reaches the lung as extremely fine zinc oxide dust. Under those circumstances, zinc in the pure metal form or as a main component of an alloy is eliminated for the combustion body according to the invention.

Magnesium can be lit with a cigarette lighter, if present as a thin wire, foil or in small granules or particles having a size below one millimeter diameter and burns with glaring brightness to completion, even if the source of ignition is removed. In this context, the ignition temperature is at 600 degrees Celsius. This also applies to magnesium alloys with small proportions of sodium, potassium or calcium, such as e.g. an alloy of 99.97 percent magnesium and 0.03 percent calcium.

Iron, if present as a compact body having a smooth surface, is very poorly ignitable. However, if iron particles with microporous surfaces are employed the situation changes. In such case the surface can exhibit reactivity so high that for example sponge iron or iron powder may ignite spontaneously in air (pyrophorous iron). The temperature at which the iron exhibits pyrophorous characteristics is adjustable within wide limits by way of the manufacturing process and by alloying with other metals. Thus, it is possible to produce pyrophorous iron for the purposes according to the invention having an ignition temperature of 250 degrees Celsius and which can be ignited with a match or cigarette lighter.

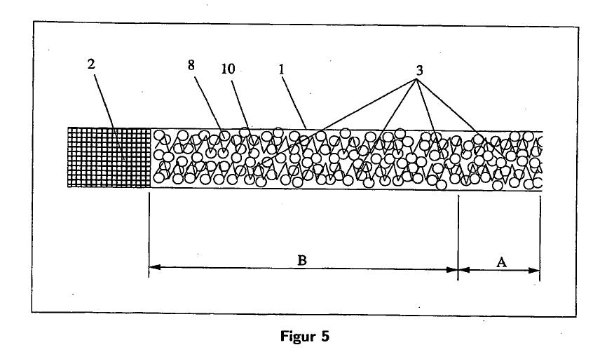

It is important for the disposable inhaler that the combustion body does not combust too rapidly. The disposable inhaler in accordance with the invention is suitable for a duration of inhalation of one to five minutes. The combustion body must continue to burn if no air is sucked through the disposable inhaler. A decisive factor for the combustion rate, the onward combustion or extinction is in this context the ratio between surface and volume of the combustion body, the heat loss to the environment as well as the amount of oxygen supplied. These parameters can be influenced in a variety of manners. In the drawings and here, in particular, in the drawing descriptions a slowing down of the combustion and brief heat storage by added granules, flakelets or little rods of silicon dioxide, calcined magnesia or ceramics is illustrated. In FIG. 5 and the associated description the combustion rate of magnesium is reduced by an enhanced oxide layer.

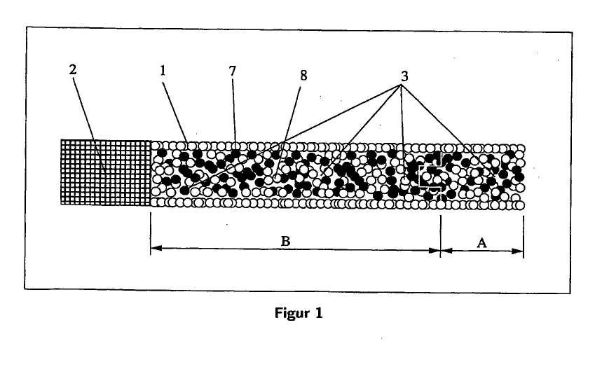

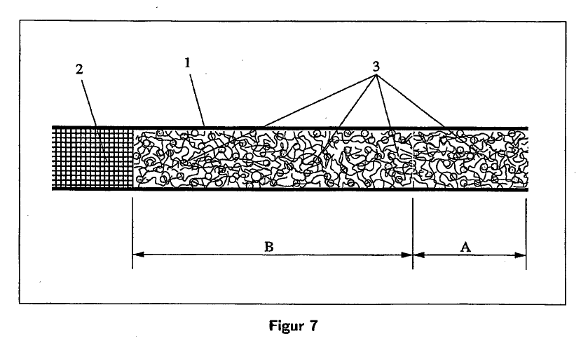

The porous, little rod-shaped combustion body, except for the end faces, is invariably enveloped in a tubular cover or coating. Metal foils, inorganic materials inert when heated (ceramics) or inorganic materials are used as enveloping material being of a kind which, when heated, will emit only water, carbon dioxide and/or oxygen. If the cover is a combustible metal foil, it will combust jointly with the combustion body to form ash as illustrated in FIGS. 2, 3, 5, 6 and 8 and in the respective drawing descriptions. If the cover is made of a material, which by heat action loses its strength, such as e.g. the cover made of magnesium carbonate bound magnesia powder in accordance with FIG. 1, this is likewise destroyed during the inhalation and forms a powderous residue. It is also possible for the cover to be kept intact during combustion. In FIG. 7 a small tube of ceramics or a cover of aluminum foil is used, which will not join in the combustion. In that case, the combustion zone progresses through the small tube. The residue being formed (ash) may be removed periodically from the small tube during inhalation.

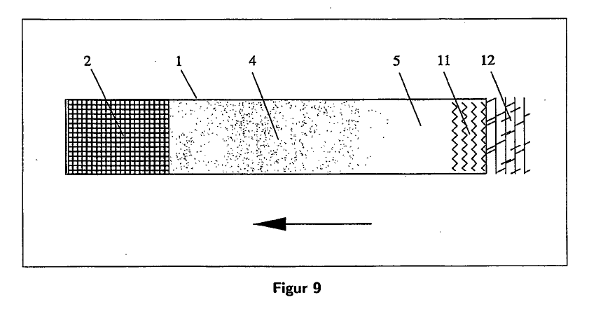

An important precondition for the desired effect of the disposable inhaler according to the invention is the prevention of pyrolysis (thermal decomposition) of the incorporated or applied active substances, because otherwise once again undesirable noxious substances—toxic carbon monoxide and/or carcinogenic substances—are formed. If the active substance is contained in the combustion body, such as in FIGS. 1, 2, 3, 4, 5 and 7, it was found to be advantageous to keep the ignition region of the combustion body free of active substance. As illustrated in FIG. 9, the combustion zone with its high temperatures will then always be in a region of the combustion body which is free of active substance.

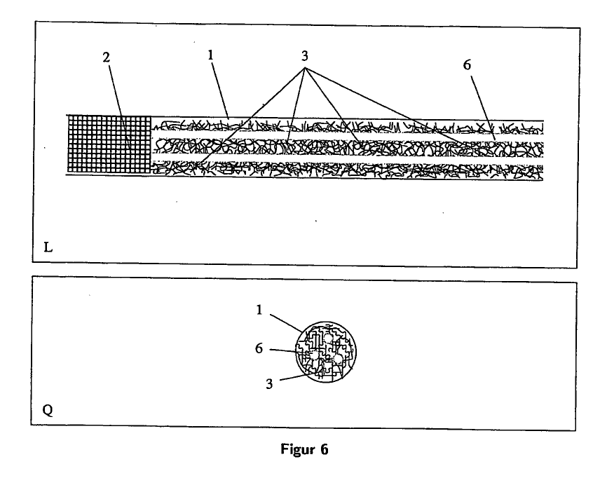

In FIG. 6 the active substance is located on the interior wall of separate small tubes passing through the combustion body. During the combustion the small tubes are heated and the partial air flow which flows through the small tubes entrains the vaporized active substance. The heat resistance of the small active substance carrier tubes takes care that the interior of the small tube attains the value of 150 degrees Celsius to 350 degrees Celsius necessary for the vaporization, but is substantially cooler than the glowing combustion zone in which temperatures between 600 degrees Celsius (filler components) and 2400 degrees Celsius (burning metal particles) prevail. Damage to the active substance by pyrolysis is thereby excluded.

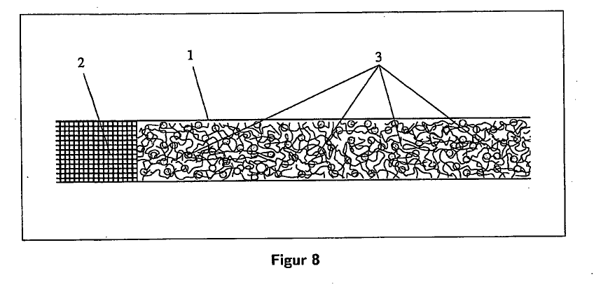

In FIG. 8 the active substance is contained in a separate active substance carrier which simultaneously acts as the filter. It is arranged downstream of the combustion body. The air heated in the combustion body has already surrendered part of the heat when it reaches the filter and has been cooled somewhat.

In the drawings FIG. 1 to FIG. 8 are working examples of the disposable inhaler according to the invention, illustrated schematically; there is shown in:

FIG. 1 the disposable inhaler in a cigarette-like configuration including magnesium granules as combustible components of the combustion body;

FIG. 2 the disposable inhaler with magnesium wool as a combustible component of the combustion body;

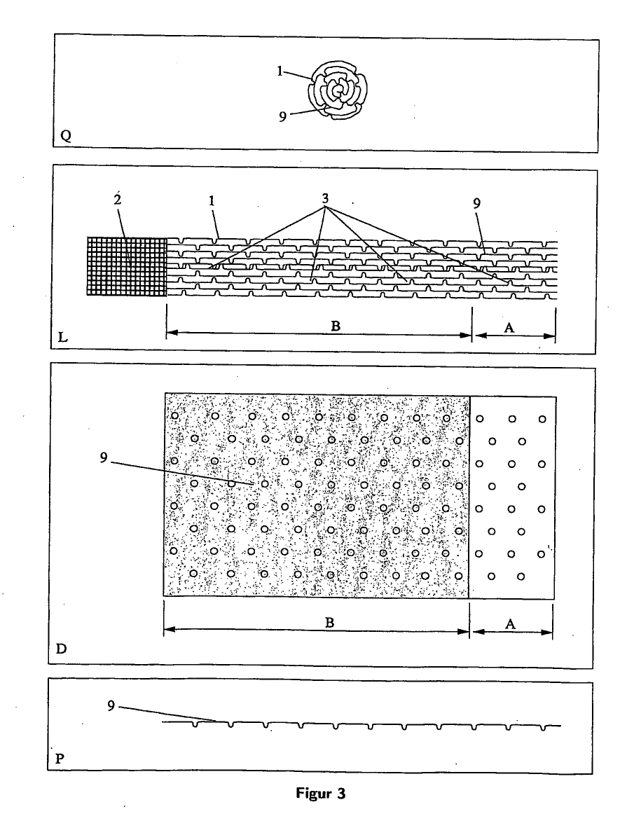

FIG. 3 the disposable inhaler with a combustion body of pyrophorous iron foil;

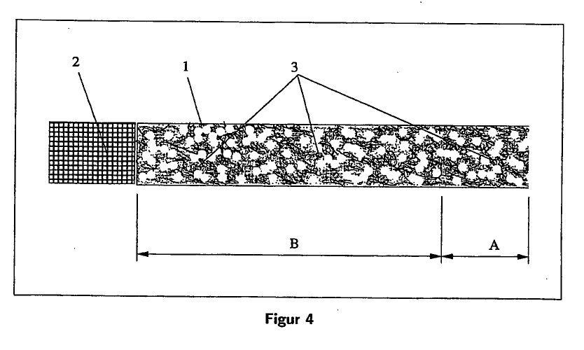

FIG. 4 the disposable inhaler with a combustion body of sponge-like structure;

FIG. 5 the disposable inhaler with a combustion body of metal spirals and ceramic granules;

FIG. 6 the disposable inhaler with the active substance in special small active substance carrier tubes;

FIG. 7 the disposable inhaler with a non-combustible or non-decomposable cover;

FIG. 8 the disposable inhaler with a separation of the combustion body and active substance carrier from one another.

FIG. 9 the mode of function of pyrolysis avoidance in disposable inhalers according to FIG. 1 to FIG. 5 and FIG. 7.

### DESCRIPTIONS OF THE EXAMPLES ILLUSTRATED IN FIG. 1 TO FIG. 8

### FIG. 1

FIG. 1 shows the disposable inhaler according to the invention in a cigarette-like configuration, comprising a cover 1, a filter 2 as well as the combustion body 3. The combustion body 3 is composed of hard calcined granules of magnesium oxide 8, granules of magnesium metal 7 and magnesium carbonate as a binder. Both types of granules are of a size of about 0.8 mm. In this example a mixture of 60 percent magnesium oxide granules and 40 percent magnesium granules is used. The granules are only connected at their contact localities by magnesium carbonate so that empty interstices remain through which air can flow. This manner of bonding is formed in that a bed in the form of the combustion body 3 is soaked with magnesium hydrogen carbonate solution and dried thereafter. During drying crystallites of magnesium carbonate, which bond the granules together, are formed from the magnesium hydrogen carbonate solution with liberation of water and carbon dioxide.

The combustion body 3 has a portion B loaded with active substance and a front portion A which is free of active substances. Both portions are manufactured separately. One portion is thinner at one end to fit into a cylindrical recess in the end face of the other part. In addition to this fit, the two parts are interconnected by the cover 1. The portion B loaded with active substance is produced in that a combustion body 3 is soaked with an organic solvent in which the active substance is dissolved, whereafter the solvent is allowed to evaporate. The cover 1 is formed in that a paste of fine magnesia powder and magnesium hydrogen carbonate solution is applied to the cylinder periphery of the two assembled portions of the combustion body 3. It solidifies when drying, similar to mortar. A conventional cigarette filter is used as a filter 2 in this example. The disposable inhaler is lit at its front end for inhalation and is then smoked like a cigarette. Due to the heat of combustion the bonding agent, magnesium carbonate, becomes destroyed with release of carbon dioxide, whereby the combustion body 3 loses its strength and the burned off parts can be flicked off as ash.

### FIG. 2

FIG. 2 shows a modification of the disposable inhaler in which the cover 1 is composed of a magnesium foil having a thickness of 0.05 mm. The combustion body 3 consists of a mixture of magnesium wool and sand (silicon dioxide). It is subdivided into a section A free of active substance and an active substance-containing section B, which are produced separately. The active substance in section B adheres to the surface of the sand particles or the metal fibers. The filter 2 in this example consists of porous sepiolith, a magnesium silicate mineral, also known as meerschaum. Both sections of the combustion body 3 and the filter 2 are held together by the enveloping foil. For inhalation the disposable inhaler is lit at its front end and then smoked like a cigarette. The fibers of the magnesium wool, jointly with the sand particles forming the combustion body 3 and the enveloping foil are combusted to form magnesium oxide, which jointly with the sand particles is stripped off in the form of ash.

### FIG. 3

FIG. 3 shows a modification of the disposable inhaler, composed of the combustion body 3, the filter 2 and the cover 1 in longitudinal section L and in transverse section Q. The combustion body 3 is formed from a wound-up iron foil 9 embossed with a nap pattern. The combustion body 3 also acts as the active substance carrier, however, the front portion of the combustion body 3 is free of active substance. The cover 1 is formed by the last wrap of the foil. Q represents the wound-up foil of the combustion body 3 in cross-section. The non-wound foil 9 with the nap pattern is shown in plan view D and in side elevation P. It has a thickness of 0.03 mm and is coated 0.5 mm thick with pyrophorous iron powder. In section B the coating carries the active substance, section A remains free of active substance. The coating is produced in that first a layer 0.3 mm thick of pyrophorous iron powder is applied onto the foil and tempered at 900 degrees Celsius in an argon atmosphere. The iron dust particles become thereby sintered together and to the foil 9. By the tempering they also lose their pyrophorous properties. In order to regenerate the latter, the layer is moistened in the presence of oxygen and allowed to rust somewhat. The iron (III) hydroxide thereby formed is subsequently reconverted into pyrophorous iron by reduction in a hydrogen atmosphere at 600 degrees Celsius. The ignition temperature of the layer is subsequently adjusted by brief tempering in the argon atmosphere at 250 degrees Celsius. The active substance is applied in section B to this coating by an active substance solution being sprayed on and the solvent being allowed to evaporate The marginal zone free of active substance on the foil later forms the active substance-free front portion of the combustion body 3. The foil 9 is eventually wound up to form a small tube. Due to the naps, interstices are formed between the windings which serve for air conduction. The disposable inhaler is lit for inhaling at its front end and then smoked like a cigarette.

### FIG. 4

FIG. 4 shows a modification of the disposable inhaler in which the combustion body 3 is formed by an open pore metal sponge of pyrophorous iron or a pyrophorous iron alloy. For its manufacture coarse magnesium granules of 2 to 3 mm diameter are compressed together with fine magnesium and iron dust at 500 degrees Celsius at a pressure of 20 MPa to form a little rod of cigarette size. In doing so, the metal particles are sintered together. Thereafter the magnesium is leached out of the structure by a sodium or potassium hydroxide solution, so that an open pore iron sponge is formed having the configuration of the desired combustion body 3. In order to render its surface pyrophorous, it is subjected to slight rusting by moistening with water in the presence of oxygen and the iron (III) hydroxide formed is subsequently reduced in a hydrogen atmosphere at 600 degrees Celsius to form pyrophorous iron. This is subsequently adjusted by brief tempering under argon to an ignition temperature of 250 degrees Celsius. The combustion body 3 consists of the active substance laden rear portion B and the active substance-free front portion A which are manufactured separately. The active substance is introduced into the pores of the section B by way of a suitable solvent. Both parts are held together jointly with the filter 2 by the cover 1, which is formed by a very thin iron foil of 0.03 mm thickness. The disposable inhaler is then lit for inhaling at its front end and smoked like a cigarette.

### FIG. 5

FIG. 5 shows a disposable inhaler, the combustion body 3 of which consists of a plurality of 0.3 mm thick, coiled magnesium wires 10 and porous ceramic particles 8 of about 1 mm diameter. The magnesium wires 10 carry an oxide layer, rendered heavier by anodizing in order to avoid combustion being too rapid. The combustion body 3, jointly with the filter 2, is held together by the cover 1, formed by a magnesium foil 0.05 mm thick. The active substance is provided in the pores and on the surface of the ceramic particles 8 of the section B. The section A is free of active substance. The magnesium wires 10, after lighting, will burn slowly from the lighting region onwards and continuously liberate the active substance in the described manner. The cover 1 and the magnesium wires 10 of the combustion body 3 are destroyed during burning down and are stripped off in the form of ash together with the ceramic active substance carrier particles 8 (which then contain no longer active substance).

### FIG. 6

FIG. 6 shows the disposable inhaler according to the invention in longitudinal section L and transverse section Q. It is composed of the cover 1, the filter 2 and the combustion body 3. The cover 1 is formed by a magnesium foil 0.05 mm thick and holds together the filter 2 and the combustion body 3. The combustion body 3 is composed of metal wool, into which the small active substance carrier tubes 6 made of magnesium carbonate bonded magnesia powder are embedded. During inhaling, part of the air flows through the metal wool of the combustion body 3 and a part flows through the hollows of the small active substance carrier tubes 6 on the interior walls of which the active substance is present as a film. The small active substance carrier tubes 6 are heated by the burning combustion body 3, as a result the active substance on the inside is evaporated and entrained by the flow of air through the small active particle carrier tube 6. The disposable inhaler is lit like a cigarette, whereafter the metal wool and the cover 1 slowly burn away. The binder of the small active substance carrier tube 6 is destroyed in the course thereof, releasing carbon dioxide so that the remaining magnesium oxide jointly with the ash of the combustion body 3 and the cover residues can be stripped off.

### FIG. 7

FIG. 7 shows a disposable inhaler, in which the cover 1 consists of a thin-walled ceramics tube or of aluminum foil. In the cover 1 a conventional cigarette filter and a combustion body 3 of metal wool are accommodated. The combustion body 3 consists of two sections, a section A free of active substance and a section B, carrying the active substance as a film on the fibers of the metal wool. The combustion body 3 is lit at the open front end of the cover 1. A combustion zone is formed which progresses slowly through the interior of the cover 1. The ash thereby formed may during the smoking procedure be removed through the front opening by tipping out. The cover 1 in this working example remains after completion of the combustion process as waste.

### FIG. 8

FIG. 8 shows a disposable inhaler, in which the entire combustion body 3 is free of active substance. It consists of a mixture of magnesium wool and sand. The active substance is deposited in the pores of the filter 2. The cover 1 is formed by a magnesium foil having a thickness of 0.05 mm. For inhaling the end of the combustion body 3 is lit. When inhaling, it slowly burns down. In the course thereof it heats the air sucked therethrough. At the beginning of the combustion process the air which reaches the filter 2 has not yet reached the vaporizing temperature, so that initially only a slight mobilization of the active substance by evaporation or sublimation takes place. The further the combustion progresses, the higher will be the temperature of the air reaching the filter 2, and accordingly the amount of entrained active substance until this has been evaporated completely.

### FIG. 9

The function diagram FIG. 9 shows that the air sucked in when inhaling initially first flows through any residual ash 12 which may be present in the combustion zone 11 of the combustion body. The combustion takes place when inhalation (smoking) commences in the front portion, which is free of active substance, of the disposable inhaler. After the air has been heated in the combustion zone, it reaches, behind the combustion zone, the portion of the combustion body 4 at this point not yet being at evaporation temperature and loaded with active substance, it evaporates the active substance and conducts the active substance vapor in the direction of the arrow to the non-burning end of the disposable inhaler. The slowly advancing combustion zone progressively reaches the regions of the combustion body 5 which no longer contain any active substance.

TABLE 1
Comparison of substances formed by the combustion of tobacco
and by the combustion of “physiological” metals.
Substances incorporated by inhalation ¹

| | From a disposable inhaler according | |
|---|---|---|
| | to the invention with nicotine | |
| | Combustion metal | Combustion metal |
| From cigarette smoke | magnesium | Fe—Ca—Zn alloy |
| 4-aminobiphenyl (1) | magnesium oxide | iron(II,III)oxide |
| acetaldehyde (2B0 | nicotine | zinc oxide |
| acetone | | calcium oxide |
| acrolein (3DF) | | nicotine |
| formic acid | | |
| ammonia | | |
| aniline (3DFG) | | |
| benz[a]anthracene (2A) | | |
| benzo(a)pyrene (2A) | | |
| benzene (1) | | |
| 1,3-butadiene (2a) | | |
| cadmium (1) | | |
| hydrogen cyanide | | |
| diethyl nitrosamine (2A) | | |
| acetic acid | | |
| ethyl methyl nitrosamine (2A) | | |
| formaldehyde (1) | | |
| hydrazine (2B) | | |
| carbon monoxide | | |
| carbon dioxide | | |
| 2-naphthylamine (1) | | |
| nickel (1) | | |
| nicotine | | |
| nitrosopyrrolidine (2B) | | |
| 2-toluidine (2A) | | |

¹ carcinogenic substances are characterized by their assessment by the IARC (International Association for Research on Cancer) stated in brackets behind the substance name. In the case of substances for which no assessment is yet available by the IARC, the assessment is given by the DFG (Deutsche Forschungsgemeinschaft). The abbreviations have the following meanings:
Assessment criteria of the IARC: 1 - “carcinogenic for humans”; 2A - “probably carcinogenic for humans”; 2B - “possibly carcinogenic for humans”.
Assessment criteria of the DFG: 1DFG - “substances which cause cancer in humans”; 2DFG - “substances which are to be considered carcinogenic for humans”; 3DFG - “substances which give reason for concern because of proven or possible carcinogenic effect”.

1. Disposable inhaler for inhaling active substances, the active substances being incorporated in an active substance carrier and being transformable by heating with hot air into the gaseous state of aggregation or an aerosol, characterized in that the heating of the drawn in air takes place by the combustion of an air-pervious combustion body, which as a combustible component contains metallic ingredients.

2. Disposable inhaler according to claim 1, characterized in that the combustion body is at the same time the active substance carrier.

3. Disposable inhaler according to claim 1, characterized in that the combustion body and the active substance carrier are different entities, which are so arranged that the air drawn in flows through the combustion body prior to flowing through the active substance carrier.

4. Disposable inhaler according to claim 2, characterized in that the lighting region of the combustion body is free of active substance.

5. Disposable inhaler according to claim 4, characterized in that the lighting region free of active substance and the active substance-laden region of the combustion body are separately manufactured units, held together by the cover 1 or by end formations fitting one into the other.

6. Disposable inhaler according to claim 1, characterized in that the combustion metal used for the heat generation is magnesium, iron, a magnesium alloy with proportions of sodium, potassium calcium, iron and/or zinc or an iron alloy with proportions of sodium, potassium, calcium, magnesium, zinc, carbon and/or phosphorus.

7. Disposable inhaler according to claim 6, characterized in that in the magnesium alloy the proportion of zinc is less than 20 percent and the proportions of sodium, potassium, calcium and/or iron/magnesium are less than 2 percent each.

8. Disposable inhaler according to claim 6, characterized in that in the iron alloy the proportion of zinc is less than 20 percent, the proportions of sodium, potassium, calcium, magnesium and/or zinc are less than 2 percent each and the proportions of carbon and/or phosphorus are less than 4 percent.

9. Disposable inhaler according to claim 1, characterized in that the combustion body is formed of metal foil, metal wire or metal wool.

10. Disposable inhaler according to claim 1, characterized in that the combustion body has added thereto substances for regulating combustion.

11. Disposable inhaler according to claim 10, characterized in that these substances are inorganic substances, inert against heat.

12. Disposable inhaler according to claim 10, characterized in that these substances are inorganic substances which on heating liberate water, carbon dioxide and/or oxygen.

13. Disposable inhaler according to claim 1, characterized in that the metallic components of the combustion body carry an oxide layer which reduces the combustion rate.

14. Disposable inhaler according to claim 1, characterized in that the combustion body consists of an air-pervious, inorganic, non-combustible structure, containing particles of combustible material.

15. Disposable inhaler according to claim 14, characterized in that the air- pervious, inorganic, non-combustible structure consists of granules, flakelets or little rods, bonded together by a bonding agent to form an air-pervious structure.
